(19) United States
(12) Patent Application Publication
(10) Pub. No.: US 2008/0166424 A1
(43) Pub. Date: Jul. 10, 2008

(54) COMPOSITIONS AND METHODS FOR TREATMENT OF DISEASES OF THE FOOT OF AN ANIMAL

(76) Inventors: Stephen P. Mixon, Montgomery, TX (US); Dennis M. Smithyman, Shreveport, LA (US); Rosemary Smithyman, Shreveport, LA (US)

Correspondence Address: SONNENSCHEIN NATH & ROSENTHAL LLP, P.O. BOX 061080, WACKER DRIVE STATION, SEARS TOWER, CHICAGO, IL, 60606-1080, US

(21) Appl. No.: 12/016,718

(22) Filed: Jan. 18, 2008

Related U.S. Application Data
(63) Continuation-in-part of application No. 11/750,223, filed on May 17, 2007.
Application No. 11/750,223 is a continuation-in-part of application No. 11/674,588, filed on Feb. 13, 2007.
Application No. 11/674,588 is a continuation of application No. 10/922,604, filed on Aug. 20, 2004, now Pat. No. 7,192,618.
(60) Provisional application No. 60/547,991, filed on Feb. 26, 2004.

(51) Int. Cl. A01N 59/16; A01N 59/20; A01N 59/06; A01N 59/02; A01P 1/00

(52) U.S. Cl. 424/618; 424/630; 424/646; 424/641; 424/682; 424/703

(57) ABSTRACT

The present invention is directed to a method for treating the foot of an animal, including treatment of infectious diseases of the foot of an animal. Among the various aspects of the invention is provided a method of contacting a foot of an animal with an antimicrobial composition comprising water, sulfuric acid, ammonium sulfate or sodium sulfate, and, optionally, an antimicrobial metal. Also provided is a method of rotational treatment wherein a foot of an animal is contacted with a series of antimicrobial composition with and without an antimicrobial metal component.

### CROSS-REFERENCE TO RELATED APPLICATIONS

This application is a continuation-in-part of U.S. application Ser. No. 11/750,223, filed on May 17, 2007, which in turn is a continuation-in-part of U.S. application Ser. No. 11/674,588, filed on Feb. 13, 2007, which in turn is a Continuation of U.S. application Ser. No. 10/922,604, filed Aug. 20, 2004, now U.S. Pat. No. 7,192,618, which in turn claims priority to U.S. Provisional Application Ser. No. 60/547,991, filed Feb. 26, 2004; each of which are incorporated herein by reference in their entirety.

### FIELD OF THE INVENTION

The present invention generally relates to compositions and methods for the treatment of an infectious disease of a foot of an animal.

### BACKGROUND OF THE INVENTION

Diseases of the foot of animals, particularly ungulates, present major health problems that can cause tremendous economic losses. Lameness in cows cause animals to lose weight and body conditioning. In dairy cows, milk production is lowered because the animals tend to go to feed less often. While diseases of the hoof are most prevalent in dairy cows, beef cattle, goats, sheep, and horses, other hoofed animals are all susceptible to these pathogens.

Common diseases of a foot of an animal, particularly the hooves of animals include hairy heel warts, foot rot, and foot scald.

Papillomatous digital dermatitis, also referred to as Hairy Heel Warts, Footwarts, and Strawberry Foot Disease, is an inflammation of the skin around the hoof of an animal. It is most commonly found at the back of the foot between the bulbs of the heels, but the disease can spread around the foot and into the cleft between the claws. Digital dermatitis is a very painful condition resulting in lameness, and subsequent reduced food intake and fertility. In dairy cows, animals infected with the disease also have reduced milk yields. Within a given herd, the proportion of animals affect by digital dermatitis can remain at low levels of less than 10% while in others, over half of the animals of a herd can be affected. The severity of the disease also varies from farm to farm.

The precise cause of digital dermatitis is not known for sure, but it is known that the disease is infectious in origin and that spiral bacteria are found in the lesions whereas they are not normally found in healthy skin. In fact, some strains of the disease are consistently found deep in damaged skin.

Foot rot, or infectious pododermatitis, is a hoof infection that is commonly found in cattle, sheep, and goats. The disease, rots away the foot of the animal, more specifically the area between the two toes of the diseased animal. Like digital dermatitis, it is extremely painful and contagious.

In cattle, the cause of foot rot is thought to be two anaerobic bacteria, Fusobacterium necrophorum and Bacteroides melaminogenicus. Both bacteria are common to the environment that cattle live in and Fusobacterium is present in the rumen and fecal matter of the cattle. The disease is also caused by the microorganism Diechelobacter nodosus which can be found in contaminated soil. Another cause of foot rot may be high temperatures or humidity causing the skin between the hooves to crack and let the bacteria infect the foot. Because of this reason, foot rot is prevalent in the southern region of the United States, where the south's wet and humid climate makes it conducive for foot rot and other diseases of the hoof.

Foot rot is also prevalent in sheep and goats and must be controlled. For instance, In a Virginia survey, approximately 21 percent of the producers considered foot rot to be a serious health problem in their flocks of sheep. As in cattle, foot rot in sheep and goats is extremely painful, contagious, and causes significant economic loss due to decreased flock production.

Foot scald, also referred to as benign foot rot or interdigital dermatitis, is an inflammation between the toes caused by F. necrophorum. Persistent moisture on the skin between the toes can increase susceptibilitiy to foot scald. Foot scald often precedes hoof rot. Like foot rot, foot scald outbreaks occur most often during persistent rainy weather along with high temperatures. If not treated these animals can become permanently infected. As with other infectious diseases of the hoof, foot scald causes stress to the animals and can affect weight gain, reproductive rates, and production. In addition, like other diseases of the hoof, Foot scald incurs additional costs to the producer for treatment and increased labor during an outbreak.

Introducing an infected animal into a non-contaminated herd can create herd contamination and therefore all animals must be treated.

Some methods currently used to treat diseases of the hoof include antibiotic treatment, chemical treatment, and antimicrobial and disinfectants.

While antibiotics can be used to rapidly reduce the severity of diseases such as digital dermatitis there are distinct disadvantages to using antibiotic therapy. For instance, the common and extensive use of antibiotics is likely to lead to the production of antibiotic resistant pathogens which are then not only problematic to the animals, but present a general concern to the public. In addition, treatment of herds with antibiotics are generally prohibitively expensive. Antibiotic therapy such as injections of long-acting penicillin or oxytetracycline, is effective, however, injections are costly and time consuming and residues may possibly be present in the milk. Although antibiotics such as oxytetracycline can be applied directly to a lesion, this becomes impractical for very large herds. While antibiotic powders can be used for treatment, and Lincomycin/spectinomcin (LS-50) powder has proven to be successful, the powder is not readily absorbed into the hoof, and therefore may not reach all areas of infection. Furthermore, topical antibiotic agents can be rapidly deactivated by organic matter and are often toxic when ingested. Again, this method of administration is not optimal for the treatment of large herds.

Chemicals have also been used to treat some of these diseases, however, caustic chemicals themselves can cause serious lameness due to chemical burns on the skin so therefore, great care must be taken in their use. For example, formalin is OSHA regulated, carcinogenic, irritating to skin, less effective in cold temperature, and potentially leaves residue in milk.

Antimicrobial agents and disinfectants are often used in the treatment of infectious diseases of the hoof. Such products often include solutions of 10 percent copper sulfate or zinc sulfate. For example, copper sulfate crystals can be used directly but are becoming increasingly expensive, leave heavy copper residues in the footbath, can contaminate the soil with high levels of copper, and are difficult to measure and store.

Footbaths currently used leave high concentrations of copper in both the soil and in the water. This is because most dairies dump the spent hoof baths into a manure pit or a lagoon so the copper ultimately gets spread on production ground with the manure. The practice can lead to copper accumulation in the soil. It is possible that, after several years, copper can accumulate in soil to levels that become toxic to soil microbes and crops. This can slow organic matter decomposition and nutrient cycling in soil and crop production could be reduced because of direct toxic effects of copper on the plants as well as reduced soil fertility. Importantly, copper accumulation in soil and forage can become toxic to sheep, whose tolerance for copper is much lower than that of dairy cattle. Toxic levels of copper in soil is a critical issue because there is no practical way to reverse the problem.

Accordingly, there is a need for compositions and methods for the treatment of infectious diseases of the feet of animals which not only exert potent antimicrobial properties, but also provide for reduction in the levels of copper required, which leads to reduced copper contamination to the soil and water and reduced costs of treatment.

### SUMMARY OF THE INVENTION

The present invention is generally directed to antimicrobial compositions for the treatment of an infectious disease of the foot of an animal.

One aspect of the invention is directed to a method for treating a foot of an animal where the method comprises contacting a foot of an animal with an antimicrobial composition comprising (a) water, (b) sulfuric acid, and (c) ammonium sulfate or sodium sulfate. In one embodiment, the antimicrobial composition comprises water, sulfuric acid, and ammonium sulfate. In another embodiment, the antimicrobial composition comprises water, sulfuric acid, and sodium sulfate.

In various embodiments, the composition further comprises an effective amount of at least one antimicrobial metal. In some configurations, the antimicrobial metal, when present, is copper, zinc, magnesium, silver, and/or iron. In one embodiment, the antimicrobial metal is copper. In another embodiment, the antimicrobial metal is zinc. In some embodiments, the antimicrobial composition further comprises an effective amount of at least two antimicrobial metals. In one embodiment, the antimicrobial composition comprises copper and zinc. In various embodiments, the antimicrobial composition comprises water, sulfuric acid, ammonium sulfate, and copper sulfate. In other embodiments, the antimicrobial composition comprises water, sulfuric acid, sodium sulfate, and copper sulfate.

In various embodiments where an antimicrobial metal is present in the antimicrobial composition, the antimicrobial metal concentration is greater than about 1 ppm. In some embodiments, the antimicrobial metal concentration is about 1 ppm to about 4,000 ppm. In some configurations, the antimicrobial metal concentration is about 10 ppm to about 300 ppm. In some configurations, the antimicrobial metal concentration is about 15 ppm to about 150 ppm. In some embodiments, the antimicrobial composition is a foam and the antimicrobial metal concentration is about 500 ppm to about 2,000 ppm. In some configurations of a foam antimicrobial composition, the antimicrobial metal concentration is about 1,000 ppm.

In various embodiments, the pH of the antimicrobial composition is about 0.5 to about 5.0. In some embodiments, the pH of the antimicrobial composition is about 1.0 to about 4.5. In some embodiments, the pH of the antimicrobial composition is about 1.5 to about 2.0 at the beginning of the treatment, the feet of at least about 300 animals are contacted with the antimicrobial composition, and the pH of the antimicrobial composition after contact with the feet of the at least about 300 animals is not more than about 4.5. In some configurations, the pH of the antimicrobial composition is about 1.5 to about 2.0 at the beginning of the treatment, the feet of at least about 400 animals are contacted with the antimicrobial composition, and the pH of the antimicrobial composition after contact with the feet of the at least about 400 animals is not more than about 4.5. In other configurations, the pH of the antimicrobial composition is about 1.5 to about 2.0 at the beginning of the treatment, the feet of at least about 500 animals are contacted with the antimicrobial composition, and the pH of the antimicrobial composition after contact with the feet of the at least about 500 animals is not more than about 4.5.

In some embodiments, the antimicrobial composition comprises water, sulfuric acid, ammonium sulfate, and copper sulfate, and the antimicrobial composition has a pH of about 1.5 to about 4.0. In other embodiments, the antimicrobial composition comprises water, sulfuric acid, sodium sulfate, and copper sulfate, and the antimicrobial composition has a pH of about 1.5 to about 4.0.

In some embodiments, the method of treatment further comprises mixing a concentrated antimicrobial composition and water to form the antimicrobial composition. In some configurations where a concentrated antimicrobial composition and water are mixed to form an antimicrobial composition, the mixed antimicrobial composition comprises an effective amount of at least one antimicrobial metal selected from the group consisting of copper, zinc, magnesium, silver, or iron at a concentration of about 10 ppm to about 300 ppm.

In some embodiments, the method of treatment further comprises mixing a concentrated antimicrobial composition, copper sulfate, and water to form the antimicrobial composition. In some configurations where a concentrated antimicrobial composition, copper sulfate, and water are mixed to form an antimicrobial composition, the mixed antimicrobial composition has a copper concentration of about 1,000 ppm to about 4,000 ppm.

In various embodiments, the treated animal is an ungulate. In some embodiments, the treated animal is a cow. In some embodiments, the treated animal is a dairy cow.

In various embodiments, contacting the foot of an animal with the antimicrobial composition occurs by bathing, spraying, or dipping. In various embodiments, the antimicrobial composition is a liquid, gel, or foam.

In various embodiments, the animal to be treated is diagnosed with, or at risk for, an infectious disease of the foot. In some embodiments, the infectious disease of the foot is hairy heel warts, foot rot, and/or foot scald.

Another aspect of the invention is directed to a method for treating a foot of an animal comprising: mixing a first concentrated antimicrobial composition and water to form a first antimicrobial composition, wherein the first concentrated antimicrobial composition comprises water, sulfuric acid, and ammonium sulfate or sodium sulfate; contacting a foot of an animal with the first antimicrobial composition; mixing a concentrated antimicrobial composition, copper sulfate, and water to form a second antimicrobial composition, wherein the second concentrated antimicrobial composition comprises water, sulfuric acid, and ammonium sulfate or sodium sulfate; and contacting the foot of the animal with the second antimicrobial composition.

Other objects and features will be in part apparent and in part pointed out hereinafter.

### BRIEF DESCRIPTION OF THE DRAWINGS

Those of skill in the art will understand that the drawings, described below, are for illustrative purposes only. The drawings are not intended to limit the scope of the present teachings in any way.

FIG. 1 is bar graph showing the number of cows with hoof lesions in two farm studies as a function of treatment with a conventional copper sulfate and formaldehyde program versus treatment with a SaniPhresh™ Step 1 and Step 2 Rotational Footbath Program. Further details regarding methodology are available in Example 1.

FIG. 2 is a bar graph showing pain scores for cows treated with a SaniPhresh™ Step 1 and Step 2 Rotational Footbath Program. Further details regarding methodology are available in Example 2.

FIG. 3 is a bar graph showing the percentage of cows wrapped in heel wart studies. Further details regarding methodology are available in Example 2.

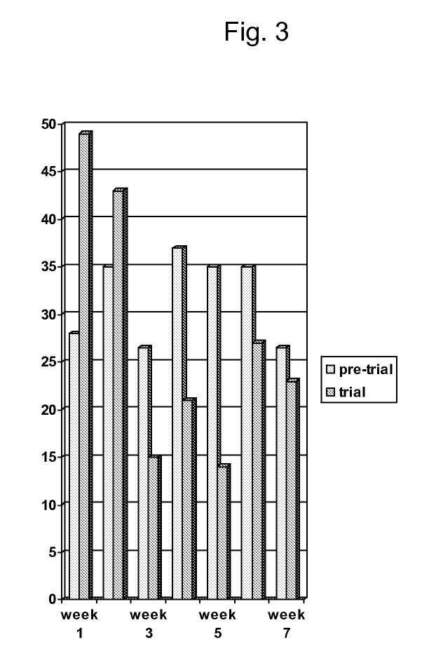

### DETAILED DESCRIPTION OF THE INVENTION

In accordance with the present invention, it has been discovered that compositions of the invention can be used to control diseases of the hoof, such as foot warts, hoof rot, and other bacterial hoof conditions more effectively than other products. Methods described herein allow decreased copper sulfate usage, increased effective levels of active bacteriostatic metals such as copper, and increased efficacy due to the various compositions described herein. Generally, the compositions for use in the methods described herein contain FDA GRAS listed ingredients and are non-toxic, safe, stable, and easy to use. Concentrated compositions described herein hold antimicrobial metals, such as copper, in solution longer for maximum effect, and use solutions penetrate sensitive areas more effectively, while not irritating the skin of an animal or human handlers. And the methods and compositions for treatment of hoof disease described herein do not require usage of antibiotics or formaldehyde. Furthermore, the methods described herein dramatically reduce heavy metal copper deposits in soil and lagoons, as compared to other available treatments.

It has been discovered that ammonium sulfate, which acts as a buffering agent, provides buffering capacity for the footbath solution and, as such, expands antimicrobial efficacy.

Thus, the present invention is directed to methods for treating a foot of an animal. The methods comprise contacting a foot of an animal with an antimicrobial composition comprising (a) water, (b) sulfuric acid, and (c) ammonium sulfate or sodium sulfate.

The present invention provides numerous benefits. As an example, upon dilution of a product such as the Unifresh® Footbath product, active ingredients salts are diluted to low levels and their benefits can be negated quickly upon spoilage of the clean footbath with, for example, manure, urine, and/or dirt. Embodiments of the composition described herein (which comprises in part, ammonium sulfate) can preserve active ingredients (e.g., copper sulfate, zinc sulfate, and sodium chloride), thus providing for higher levels of active ingredients in the footbath and longer lasting effects. Various embodiments of the activator composition described herein have been found to maintain effective levels of antimicrobial and hoof conditioning benefits of the footbath for up to, for example, 300 cows, 400 cows, 500 cows, or more, which can depend on various factors including, but not limited to, water quality, farm cleanliness and footbath management.

Another realized benefit of the antimicrobial composition is the maintenance of low levels of digital dermatitis (i.e., heel warts) and interdigital dermatitis (i.e., heel erosion and/or foot rot) with routine use. The antimicrobial composition of the invention, when used in a regular footbath program, helps to promote hoof health. Healthy hooves translate to a reduction in hoof diseases and maintenance of a low incidence of these diseases in the herd. This in turn can prevent the tremendous economic losses discussed above, which can occur when animals are afflicted with these diseases.

In addition to the efficacy of antimicrobial compositions, environmental benefits can be appreciated with the present invention due to the reduction in the amount of antimicrobial metal (e.g., copper and/or zinc) used in the footbaths, as compared to conventional treatment protocols. As described herein, the amount of effective antimicrobial metal is increased through, at least in part, the buffering capacity of the composition. Because there can be reduction in the amount of metal in the footbath used, there can be consequently a reduction in the amount of metal transferred to the soil and to the water. Exemplary antimicrobial compositions, such as Tasker Unifresh® and SaniPhresh™ footbath products, typically comprise about 12 pounds of copper sulfate pentahydrate per 50 gallons (corresponding to about 3 pounds of copper per 50 gallons). This represents a reduction in the amount of copper sulfate used in conventional treatment programs.

The benefits of the present invention, which include efficacious non-toxic antimicrobial compositions, maintenance of low levels of infectious diseases, and/or reduced amounts of copper being transferred to the soil and water, make the use of the antimicrobial composition superior to other antimicrobial footbaths known in the art.

Composition

The present invention is generally directed to compositions and methods for the treatment of infectious diseases of a foot of an animal, particularly the foot/hoof of an ungulate animal. The antimicrobial composition can be used, for example, in a concentrated form, a diluted form, or as a powder.

In various embodiments, the antimicrobial compositions are bacteriostatic agents that are non-toxic and composed of FDA GRAS materials. The disinfection compositions and methods of the present invention have activity against a wide variety of microorganisms such as Gram positive and Gram negative bacteria, yeast, molds, bacterial spores, viruses, etc. The antimicrobial composition can provide antimicrobial effects against microbes including, but not limited to, Fusobacterium necrophorum, Bacteroides melaminogenicus, Dichelobacter nodosus, Staphylococcus intermedius, Streptococcus mutans, Escherichia coli, Staphylococcus aureus, Listeria monocytogenes, Salmonella typhimurium, Salmonella (nontyphoidal), Pseudomonas, Vibrio parahaemolyticus, V. vulnificus, V. mimicus, V. fluvialis, V. furnissii, V. hollisae, V. cholerae, (and other Vibrio spp.), Bacillus cereus, Clostridium botulinum, Clostridium perfringens, Campylobacter jejuni, Shigella, and the like (see e.g., Examples 1-4). The methods described herein particularly benefit from the composition's antimicrobial effects against Fusobacterium necrophorum, Bacteroides melaminogenicus, and Diechelobacter nodosus (see e.g., Examples 1-2).

In various embodiments, the antimicrobial composition of the invention is a buffered acidic disinfection composition. The disinfection composition can be a buffered acidic solution of an acid and a salt of an acid and a base (e.g., Tasker Unifresh). Exemplary acidic agents include those provided in Table 1. Exemplary buffering systems include corresponding salts. In some embodiments, the antimicrobial composition comprises water, sulfuric acid, and ammonium sulfate. In some embodiments, the antimicrobial composition comprises water, sulfuric acid, and sodium sulfate. In some embodiments, the antimicrobial composition comprises water, sulfuric acid, ammonium sulfate, and sodium sulfate.

For example, a buffered acidic disinfection composition for use in the methods described herein can be formed by reacting 98% sulfuric acid with a 13-18% by weight ammonium sulfate in water solution (order of addition is ammonium sulfate solution to sulfuric acid) at approximately 300-350° F. for 24 hours, where electrolysis of the reacting solution is applied for 1 hour at the start of the process, with a stabilization step (addition of more ammonium sulfate solution to ensure that the reaction is complete) after overnight cooling. As another example, the same process can be performed but at approximately 200-210° F. for 2 hours with a stabilization step immediately after the 1 hour electrolysis period. As a further example, a buffered acidic disinfection composition for use in the methods described herein can be formed, in a “cold process”, by adding 98% sulfuric acid slowly to a 30% by weight ammonium sulfate solution, with no stabilization step, at a temperature of 150-200° F. during the addition process. As yet another example, a buffered acidic disinfection composition for use in the methods described herein can be formed by reacting 98% sulfuric acid with a 13-18% by weight sodium sulfate in water (order of addition is sodium sulfate solution to sulfuric acid) for 4 hours at approximately 300-350° F. with a stabilization step (addition of more sodium sulfate solution to ensure that the reaction is complete) after cooling, where electrolysis of the reacting solution is applied for 1 hour at the start of the process. In still another example, a buffered acidic disinfection composition for use in the methods described herein can be formed, in a “cold process” (i.e., no electrolysis step), by reacting 98% sulfuric acid with a 26-28% by weight sodium sulfate in water solution for 4 hours at approximately 300-350° F. with a stabilization step after cooling.

The antimicrobial composition can comprise the compositions described in U.S. Pat. No. 5,989,595; U.S. Pat. No. 6,242,011; U.S. Pat. No. 7,192,618, each of which are incorporated herein by reference in their entirety. The antimicrobial composition can comprise the compositions described in U.S. Patent App. Pub. No. 2005/0191365; U.S. Patent App. Pub. No. 2005/0191394; U.S. Patent App. Pub. No. 2005/0191395; and U.S. Patent App. Pub. No. 2006/0228308, each of which are incorporated herein by reference in their entirety. The antimicrobial composition can comprise the compositions described in U.S. patent application Ser. No. 11/674,588; U.S. application Ser. No. 11/874,034; and U.S. application Ser. No. 11/750,223, each of which are incorporated herein by reference in their entirety. One of skill in the art will understand that the concentrated antimicrobial composition can be diluted to a use concentration.

The antimicrobial composition can also contain an antimicrobial metal. In some embodiments, the antimicrobial composition is a buffered acidic agent in combination with an antimicrobial metal-containing agent capable of providing free metal ions in solution (see U.S. Patent App. Pub. No. 2005/0191365, incorporated herein by reference). In some embodiments, the antimicrobial metal of the composition is copper, zinc, magnesium, silver, and/or iron. The antimicrobial composition can contain at least one antimicrobial metal, at least two antimicrobial metals, at least three antimicrobial metals, or at least four antimicrobial metals.

The antimicrobial metal(s) are generally present in amount effective to provide an antimicrobial effect. It is contemplated that where multiple antimicrobial metals are present, each can be present in an antimicrobial effective amount; at least one metal is present in an antimicrobial effective amount; or none of the antimicrobial metals are present in an antimicrobial effective amount when considered individually but together provide for such effect. In some embodiments, the antimicrobial composition contains an antimicrobial metal at a concentration of at least about 1 ppm. In some embodiments, an antimicrobial metal is present in an antimicrobial composition at a concentration of up to about 4,000 ppm. In some embodiments, an antimicrobial composition for contact with the foot of an animal contains about 10 to about 300 ppm of an antimicrobial metal. For example, an antimicrobial metal can be present in an antimicrobial composition at a concentration of about 10 ppm, about 20 ppm, about 30 ppm, about 40 ppm, about 50 ppm, about 60 ppm, about 70 ppm, about 80 ppm, about 90 ppm, about 100 ppm, about 110 ppm, about 120 ppm, about 130 ppm, about 140 ppm, about 150 ppm, about 160 ppm, about 170 ppm, about 180 ppm, about 190 ppm, about 200 ppm, about 210 ppm, about 220 ppm, about 230 ppm, about 240 ppm, about 250 ppm, about 260 ppm, about 270 ppm, about 280 ppm, about 290 ppm, or about 300 ppm. Preferably, an antimicrobial composition for contact with the foot of an animal, formed from a concentrate antimicrobial composition (examples of which are described above) can contain about 15 ppm to about 150 ppm, and more preferably, about 30 ppm to about 75 ppm.

In some embodiments, antimicrobial metal can be provided at elevated levels of up to about 4,000 ppm. As an example, such elevated levels can be accomplished in an aqueous composition with a two step process by adding additional antimicrobial metal to an aqueous antimicrobial composition formed form a concentrated antimicrobial composition or from a powder formulation. As another example, a foam or gel antimicrobial composition can contain about 500 ppm to about 2,000 ppm, preferably about 1,000 ppm, of an antimicrobial metal(s) (e.g., copper, zinc, or copper and zinc). Generally, an antimicrobial metal can be supplied in the antimicrobial compositions described herein in a variety of forms, preferably in a water soluble form.

In some embodiments where a concentrated antimicrobial composition is provided, the antimicrobial metal concentration can range from about 0.1 wt % to about 5 wt %. As an example, a concentrated antimicrobial composition can contain about 1.0 wt % to about 3.0 wt % copper. As another example, a concentrated antimicrobial composition can contain about 0.5 wt % to about 1.5 wt % zinc. As another example, a concentrated antimicrobial composition can contain about 1.0 wt % to about 3.0 wt % copper and about 0.5 wt % to about 1.5 wt % zinc.

In some embodiments, elevated concentrations of antimicrobial metal can be present in the antimicrobial composition for contact with the foot of an animal. As indicated above, the elevated concentrations of antimicrobial metal can be as high as 4,000 ppm, or even greater. In some embodiments, additional antimicrobial metal can be added to an antimicrobial solution formed from a concentrated antimicrobial solution. As an example, an antimicrobial composition for contact with the foot of an animal containing about 10 ppm to about 300 ppm of an antimicrobial metal can be formed from a concentrated antimicrobial solution, with additional antimicrobial metal being added thereto. For example, antimicrobial copper (e.g., copper sulfate pentahydrate) can be added to an antimicrobial composition containing about 10 ppm to about 300 ppm copper so as to bring the concentration to an elevated level such as about 1,000 ppm, about 2,000 ppm, about 3,000 ppm, or about 4,000 ppm copper. In some configurations, about 10 to about 15 pounds of copper sulfate pentahydrate is added to a footbath containing about 10 ppm to about 300 ppm so as to bring the final copper (Cu²⁺) concentration of the antimicrobial composition footbath to about 3,000 ppm or more.

Preferably, the antimicrobial metal is copper, or copper in combination with another antimicrobial metal. In some embodiments, an antimicrobial composition can contain both copper and zinc, present in antimicrobial effective amounts individually or in combination. In some embodiments, the antimicrobial composition contains copper at a concentration of at least about 1 ppm. In some embodiments, the antimicrobial composition contains about 1 ppm to about 300 ppm copper. For example, the antimicrobial composition can contain about 15 ppm to about 200 ppm copper. Preferably, the antimicrobial composition contains about 30 ppm to about 150 ppm copper.

Also as discussed above, in some embodiments, antimicrobial copper can be provided at elevated levels of up to about 4,000 ppm. As an example, such elevated levels can be accomplished in an aqueous composition with a two step process by adding additional copper (e.g., in the form of a copper sulfate such as copper sulfate pentahydrate) to an aqueous antimicrobial composition formed form a concentrated antimicrobial composition or from a powder formulation. As another example, a foam or gel antimicrobial composition can contain about 500 ppm to about 2,000 ppm, preferably about 1,000 ppm, of an antimicrobial metal(s) (e.g., copper, zinc, or copper and zinc). Generally, an antimicrobial metal can be supplied in the antimicrobial compositions described herein in a variety of forms. For example, copper can be supplied as copper sulfate, preferably, copper sulfate pentahydrate.

In some embodiments, the antimicrobial composition contains zinc at a concentration of at least about 1 ppm. In some embodiments, the antimicrobial composition contains about 1 ppm to about 300 ppm zinc. For example, the antimicrobial composition can contain about 10 ppm to about 150 ppm zinc. Preferably, the antimicrobial composition contains about 15 ppm to about 75 ppm zinc. In some embodiments, an antimicrobial composition can contain both copper and zinc, present in antimicrobial effective amounts individually or in combination. Zinc can be supplied in the antimicrobial compositions described herein in a variety of forms such as zinc sulfate, preferably, zinc sulfate monohydrate.

Preferably, the antimicrobial composition is a buffered acidic agent in combination with at least a copper containing agent capable of providing free copper ions in solution. Examples of various copper-containing agents include copper metal (inorganic copper), cuprous sulfate, cupric sulfate, and copper sulfate pentahydrate. In some embodiments, the antimicrobial composition comprises water, sulfuric acid, ammonium sulfate, and copper sulfate. In some embodiments, the antimicrobial composition comprises water, sulfuric acid, sodium sulfate, and copper sulfate. In some embodiments, the antimicrobial composition comprises water, sulfuric acid, ammonium sulfate, sodium sulfate, and copper sulfate. The copper-containing buffered acidic antimicrobial composition for use in the methods described herein can be formed by the addition of various forms of copper to the various forms of acidic buffered antimicrobial composition described above.

In some embodiments, the antimicrobial composition is an acidic agent in combination with a buffer, a sulfate-containing agent, and at least one antimicrobial metal agent (preferably copper). In some embodiments, a single agent can deliver both metal ions and sulfate, for example copper sulfate and/or zinc sulfate. Such a mixture produces a copper sulfate complex that is highly protonated and at a low pH. Further, the sulfate component is thought to enhance antimicrobial metal and proton uptake by microbes. For example, a copper-containing buffered acidic antimicrobial composition, also containing sulfate, can be formed by mixing water (about 68%), one of the acidic buffered antimicrobial compositions described above (about 12%), and copper sulfate or copper sulfate pentahydrate (about 20%). In some configurations, the antimicrobial solution is Tasker SaniPhresh™. The low pH (buffered inorganic acidic) solution can serve as the active (e.g., ionic Cu2+ form) carrier of copper.

An antimicrobial composition can be used at a range of effective concentrations. In some embodiments, an antimicrobial composition has a pH of about 0.5 to about 5.0. In some embodiments, an antimicrobial composition has a pH of about 1.0, about 1.5, about 2.0, about 2.5, about 3.0, about 3.5, about 4.0, or about 4.5. Preferably, an aqueous antimicrobial composition has a pH of about 1.5 to 2.0 upon initial treatment; as treatment progresses, the pH can increase through at least about 4.5 and remain effective. Preferably, a foam or gel antimicrobial composition has a pH of about 0.5 to about 1.5, more preferably, about 1.0. In some embodiments, an antimicrobial composition comprises an aqueous solution of water, sulfuric acid, and sodium sulfate and/or copper sulfate, and the antimicrobial composition has a pH of about 1.0 to about 4.5. The antimicrobial composition can be modified for increased buffering so as to further increase the pH at which the composition retains effectiveness. Various additives to increase buffering capacity and extend effective pH range are as described in U.S. Prov. App. Ser. No. 60/982,494, incorporated herein by reference in its entirety. As an example, the concentration of zinc can be increased so as to extend the effective pH range of an antimicrobial composition.

The various metal-containing buffered acidic antimicrobial compositions can be used in combination with additional buffered acidic antimicrobial compositions to achieve the prescribed pH control and copper content of the disinfection composition. For example, a buffered acidic antimicrobial composition can be used for pH control, while a metal-containing buffered acidic antimicrobial composition can be used for antimicrobial metal control—these products can be added separately or in a pre-formulated blend to achieve a desired pH range and/or antimicrobial metal range. Such products can also be used sequentially in a rotating program. Water testing can be performed to determine the concentrations to add to achieve the desired targets.

It can be desirable that each of the antimicrobial composition ingredients are generally recognized as safe (GRAS) and are permitted for use as direct human food ingredients using good manufacturing practice. Exemplary GRAS acids are provided in Table 1.

TABLE 1
Acids Generally Recognized as Safe (GRAS)

| Acid Name | CAS No. |
|---|---|
| ACETIC ACID | 000064-19-7 |
| ACONITIC ACID | 000499-12-7 |
| ADIPIC ACID | 000124-04-9 |
| ALGINIC ACID | 009005-32-7 |
| P-AMINOBENZOIC ACID | 000150-13-0 |
| AMINO TRI(METHYLENE PHOSPHONIC ACID), SODIUM | 020592-85-2 |
| SALT | |
| ANISIC ACID | 001335-08-6 |
| ASCORBIC ACID | 000050-81-7 |
| L-ASPARTIC ACID | 000056-84-8 |
| BENZOIC ACID | 000065-85-0 |
| N-BENZOYLANTHRANILIC ACID | 000579-93-1 |
| BORIC ACID | 010043-35-3 |
| (E)-2-BUTENOIC ACID | 003724-65-0 |
| BUTYRIC ACID | 000107-92-6 |
| CHOLIC ACID | 000081-25-4 |
| CINNAMIC ACID | 000621-82-9 |
| CITRIC ACID | 000077-92-9 |
| CYCLOHEXANEACETIC ACID | 005292-21-7 |
| CYCLOHEXANECARBOXYLIC ACID | 000098-89-5 |
| DECANOIC ACID | 000334-48-5 |
| 5-DECENOIC ACID | 085392-03-6 |
| 6-DECENOIC ACID | 085392-04-7 |
| 9-DECENOIC ACID | 014436-32-9 |
| (E)-2-DECENOIC ACID | 000334-49-6 |
| 4-DECENOIC ACID | 026303-90-2 |
| DEHYDROACETIC ACID | 000520-45-6 |
| DESOXYCHOLIC ACID | 000083-44-3 |
| 2,4-DIHYDROXYBENZOIC ACID | 000089-86-1 |
| 3,7-DIMETHYL-6-OCTENOIC ACID | 000502-47-6 |
| 2,4-DIMETHYL-2-PENTENOIC ACID | 066634-97-7 |
| ERYTHORBIC ACID | 000089-65-6 |
| 2-ETHYLBUTYRIC ACID | 000088-09-5 |
| 4-ETHYLOCTANOIC ACID | 016493-80-4 |
| FOLIC ACID | 000059-30-3 |
| FORMIC ACID | 000064-18-6 |
| FUMARIC ACID | 000110-17-8 |
| GERANIC ACID | 000459-80-3 |
| GIBBERELLIC ACID | 977136-81-4 |
| D-GLUCONIC ACID | 000526-95-4 |
| L-GLUTAMIC ACID | 000056-86-0 |
| GLUTAMIC ACID HYDROCHLORIDE | 000138-15-8 |
| GLYCOCHOLIC ACID | 000475-31-0 |
| HEPTANOIC ACID | 000111-14-8 |
| (E)-2-HEPTENOIC ACID | 018999-28-5 |
| HEXANOIC ACID | 000142-62-1 |
| TRANS-2-HEXENOIC ACID | 013419-69-7 |
| 3-HEXENOIC ACID | 004219-24-3 |
| HYDROCHLORIC ACID | 007647-01-0 |
| 4-HYDROXYBENZOIC ACID | 000099-96-7 |
| 4-HYDROXYBUTANOIC ACID LACTONE | 000096-48-0 |
| 4-HYDROXY-2-BUTENOIC ACID GAMMA-LACTONE | 000497-23-4 |
| 5-HYDROXY-2,4-DECADIENOIC ACID DELTA-LACTONE | 027593-23-3 |
| 5-HYDROXY-2-DECENOIC ACID DELTA-LACTONE | 051154-96-2 |
| 5-HYDROXY-7-DECENOIC ACID DELTA-LACTONE | 025524-95-2 |
| 4-HYDROXY-2,3-DIMETHYL-2,4-NONADIENOIC ACID | 000774-64-1 |
| GAMMA LACTONE | |
| 6-HYDROXY-3,7-DIMETHYLOCTANOIC ACID LACTONE | 000499-54-7 |
| (Z)-4-HYDROXY-6-DODECENOIC ACID LACTONE | 018679-18-0 |
| 5-HYDROXY-2-DODECENOIC ACID LACTONE | 016400-72-9 |
| 1-HYDROXYETHYLIDENE-1,1-DIPHOSPHONIC ACID | 002809-21-4 |
| 2-(2-HYDROXY-4-METHYL-3-CYCLOHEXENYL)PROPIONIC | 057743-63-2 |
| ACID GAMMA-LACTONE | |
| 4-HYDROXY-4-METHYL-7-CIS-DECANOIC ACID | 070851-61-5 |
| GAMMALACTONE | |
| 5-HYDROXY-4-METHYLHEXANOIC ACID DELTA-LACTONE | 010413-18-0 |
| 4-HYDROXY-4-METHYL-5-HEXENOIC ACID GAMMA | 001073-11-6 |
| LACTONE | |
| 4-HYDROXY-3-METHYLOCTANOIC ACID LACTONE | 039212-23-2 |
| HYDROXYNONANOIC ACID, DELTA-LACTONE | 003301-94-8 |
| 3-HYDROXY-2-OXOPROPIONIC ACID | 001113-60-6 |
| 4-HYDROXY-3-PENTENOIC ACID LACTONE | 000591-12-8 |
| 5-HYDROXYUNDECANOIC ACID LACTONE | 000710-04-3 |
| 5-HYDROXY-8-UNDECENOIC ACID DELTA-LACTONE | 068959-28-4 |
| ISOBUTYRIC ACID | 000079-31-2 |
| ISOVALERIC ACID | 000503-74-2 |
| ALPHA-KETOBUTYRIC ACID | 000600-18-0 |
| LACTIC ACID | 000050-21-5 |
| LAURIC ACID | 000143-07-7 |
| LEVULINIC ACID | 000123-76-2 |
| LIGNOSULFONIC ACID | 008062-15-5 |
| LINOLEIC ACID | 000060-33-3 |
| L-MALIC ACID | 000097-67-6 |
| MALIC ACID | 000617-48-1 |
| 2-MERCAPTOPROPIONIC ACID | 000079-42-5 |
| 2-METHOXYBENZOIC ACID | 000579-75-9 |
| 3-METHOXYBENZOIC ACID | 000586-38-9 |
| 4-METHOXYBENZOIC ACID | 000100-09-4 |
| TRANS-2-METHYL-2-BUTENOIC ACID | 000080-59-1 |
| 2-METHYLBUTYRIC ACID | 000116-53-0 |
| 3-METHYLCROTONIC ACID | 000541-47-9 |
| 2-METHYLHEPTANOIC ACID | 001188-02-9 |
| 2-METHYLHEXANOIC ACID | 004536-23-6 |
| 5-METHYLHEXANOIC ACID | 000628-46-6 |
| 4-METHYLNONANOIC ACID | 045019-28-1 |
| 4-METHYLOCTANOIC ACID | 054947-74-9 |
| 3-METHYL-2-OXOBUTANOIC ACID | 000759-05-7 |
| 3-METHYL-2-OXOPENTANOIC ACID | 001460-34-0 |
| 4-METHYL-2-OXOPENTANOIC ACID | 000816-66-0 |
| 3-METHYLPENTANOIC ACID | 000105-43-1 |
| 4-METHYLPENTANOIC ACID | 000646-07-1 |
| 2-METHYL-2-PENTENOIC ACID | 003142-72-1 |
| 2-METHYL-3-PENTENOIC ACID | 037674-63-8 |
| 2-METHYL-4-PENTENOIC ACID | 001575-74-2 |
| 4-METHYLPENT-2-ENOIC ACID | 010321-71-8 |
| 3-METHYL-3-PHENYL GLYCIDIC ACID ETHYL ESTER | 000077-83-8 |
| 4-(METHYLTHIO)-2-OXOBUTANOIC ACID | 000583-92-6 |
| 2-METHYLVALERIC ACID | 000097-61-0 |
| MYRISTIC ACID | 000544-63-8 |
| NITRIC ACID | |
| NONANOIC ACID | 000112-05-0 |
| (E)-2-NONENOIC ACID | 014812-03-4 |
| 2-NONENOIC ACID GAMMA-LACTONE | 021963-26-8 |
| 9,12-OCTADECADIENOIC ACID (48%) AND 9,12,15- | 977043-76-7 |
| OCTADECATRIENOIC ACID (52%) | |
| OCTANOIC ACID | 000124-07-2 |
| (E)-2-OCTENOIC ACID | 001871-67-6 |
| OLEIC ACID | 000112-80-1 |
| 3-OXODECANOIC ACID GLYCERIDE | 128331-45-3 |
| 3-OXODODECANOIC ACID GLYCERIDE | 128362-26-5 |
| 3-OXOHEXADECANOIC ACID GLYCERIDE | 128331-46-4 |
| 3-OXOHEXANOIC ACID DIGLYCERIDE | 977148-06-3 |
| 3-OXOOCTANOIC ACID GLYCERIDE | 128331-48-6 |
| 2-OXOPENTANEDIOIC ACID | 000328-50-7 |
| 2-OXO-3-PHENYLPROPIONIC ACID | 000156-06-9 |
| 3-OXOTETRADECANOIC ACID GLYCERIDE | 128331-49-7 |
| PALMITIC ACID | 000057-10-3 |
| 4-PENTENOIC ACID | 000591-80-0 |
| 2-PENTENOIC ACID | 013991-37-2 |
| PERACETIC ACID | 000079-21-0 |
| PERIODIC ACID | 010450-60-9 |
| PHENOXYACETIC ACID | 000122-59-8 |
| PHENYLACETIC ACID | 000103-82-2 |
| 3-PHENYLPROPIONIC ACID | 000501-52-0 |
| PHOSPHORIC ACID | 007664-38-2 |
| POLY(ACRYLIC ACID-CO-HYPOPHOSPHITE), SODIUM SALT | 071050-62-9 |
| POLYACRYLIC ACID, SODIUM SALT | 009003-04-7 |
| POLYMALEIC ACID | 026099-09-2 |
| POLYMALEIC ACID, SODIUM SALT | 030915-61-8 |
| POTASSIUM ACID PYROPHOSPHATE | 014691-84-0 |
| POTASSIUM ACID TARTRATE | 000868-14-4 |
| PROPIONIC ACID | 000079-09-4 |
| 2-(4-METHYL-2-HYDROXYPHENYL)PROPIONIC ACID- | 065817-24-5 |
| GAMMA-LACTONE | |
| PYROLIGNEOUS ACID | 008030-97-5 |
| PYRUVIC ACID | 000127-17-3 |
| SALICYLIC ACID | 000069-72-7 |
| SODIUM ACID PYROPHOSPHATE | 007758-16-9 |
| SODIUM BISULFATE (SODIUM ACID SULFATE) | |
| SORBIC ACID | 000110-44-1 |
| STEARIC ACID | 000057-11-4 |
| SUCCINIC ACID | 000110-15-6 |
| SULFAMIC ACID | 005329-14-6 |
| SULFURIC ACID | 007664-93-9 |
| SULFUROUS ACID | 007782-99-2 |
| TANNIC ACID | 001401-55-4 |
| TARTARIC ACID, L | 000087-69-4 |
| TAUROCHOLIC ACID | 000081-24-3 |
| 1,2,5,6-TETRAHYDROCUMINIC ACID | 056424-87-4 |
| THIOACETIC ACID | 000507-09-5 |
| THIODIPROPIONIC ACID | 000111-17-1 |
| TRIFLUOROMETHANE SULFONIC ACID | 001493-13-6 |
| (2,6,6-TRIMETHYL-2-HYDROXYCYCLOHEXYLIDENE)ACETIC | 015356-74-8 |
| ACID GAMMA-LACTONE | |
| UNDECANOIC ACID | 000112-37-8 |
| 10-UNDECENOIC ACID | 000112-38-9 |
| N-UNDECYLBENZENESULFONIC ACID | 050854-94-9 |
| VALERIC ACID | 000109-52-4 |
| VANILLIC ACID | 000121-34-6 |

Compositions described above can be produced in accord with the methods and formulations as described in U.S. Pat. No. 5,989,595; U.S. Pat. No. 6,242,011; U.S. Pat. No. 7,192,618; U.S. Patent App. Pub. No. 2005/0191365; U.S. Patent App. Pub. No. 2005/0191394; U.S. Patent App. Pub. No. 2005/0191395; U.S. Patent App. Pub. No. 2006/0228308; U.S. Patent App. Pub. No. 2007/0264398; U.S. Patent App. Pub. No. 2007/0269563; and U.S. application Ser. No. 11/874,034, incorporated herein by reference.

Generally, an effective acidic copper containing antimicrobial composition can be made by combining an acid, a buffer, and a copper containing substance so as to reach a desired pH and copper concentration (e.g., pH of about 0.5 to about 5.0, preferably about 1.0 to about 4.5, more preferably about 1.5 to about 2.0; and a copper concentration of about 1 ppm to about 4,000 ppm, preferably about 10 ppm to about 300 ppm, more preferably about 75 ppm to about 150 ppm). For example, an acid, a buffer, and a copper containing substance can be combined in equal measure in a vessel at room temperature so as to reach a pH of about 1.5 to about 2.0 and a copper concentration of about 75 ppm to about 150 ppm. Where the antimicrobial composition comprises an acidic buffered composition, the actual application requirement can be a function of the alkalinity of the added water. The antimicrobial composition can be titrated until reaching the target pH, then monitored and maintained. As described above, the pH and antimicrobial metal concentration can be independently controlled.

In various embodiments, the antimicrobial composition comprises sulfuric acid, ammonium sulfate, and copper sulfate, and can optionally further comprise zinc sulfate, sodium chloride, and potassium chloride.

Various embodiments of a concentrated form of the antimicrobial composition described herein can include about 30% to about 80% water; about 12% to about 28% sulfuric acid; at least about 0% to about 20% ammonium sulfate, about 3% to about 18% copper sulfate; about 0.1% to about 10% zinc sulfate; about 0.1% to about 5% sodium chloride; and about 0.1% to about 5% potassium chloride. Preferably, an antimicrobial composition includes about 60% to about 70% water; about 15% to about 25% sulfuric acid; at least about 0% to about 10% ammonium sulfate; about 5% to about 15% copper sulfate; about 1% to about 5% zinc sulfate; about 0.1% to about 1.5% sodium chloride; and about 0.1% to about 1.5% potassium chloride.

As an example, a concentrated antimicrobial composition can include: about 50% to about 75% water, about 15% to about 25% sulfuric acid; about 5% to about 15% copper sulfate; about 1% to about 9% ammonium sulfate; about 1% to about 5% zinc sulfate; about 0.1% to about 1% sodium chloride; and about 0.1% to about 1% potassium chloride (e.g., Tasker Unifresh®). In a preferred embodiment, the antimicrobial composition includes 69.0% by weight of water; 20.5% by weight of 93% Sulfuric Acid; 5.0% by weight copper sulfate; 3.0% by weight zinc sulfate; 1.0% by weight sodium chloride; 1.0% by weight potassium chloride; 0.5% by weight ammonium sulfate (e.g., Tasker Unifresh® FBV2 modified with 2.9% Tasker Clear). In another preferred embodiment, the antimicrobial composition includes 62.8% by weight water; 20.5% by weight of 93% sulfuric Acid; 9.1% by weight copper sulfate; 5.8% by weight ammonium sulfate; 1.2% by weight zinc sulfate; 0.3% by weight sodium chloride; and 0.3% by weight potassium chloride (e.g., Unifresh® FBV2 modified with 34.6% by weight of Tasker Clear and 45.5% by weight Tasker Blue). The concentrated antimicrobial composition can be used to form a diluted use antimicrobial composition by, for example, adding about 0.1 ounces to about 1 ounce of concentrate to about one gallon of water. For example, various concentrated antimicrobial compositions discussed above can be used to form a diluted use antimicrobial composition by adding about 0.4 ounces to about 0.64 ounces of concentrate to about one gallon of water (i.e., about 20 to about 32 ounces of concentrate per about 50 gallons). Preferably, about 0.5 ounces of concentrate described above is added to about one gallon of water. The actual amount added can vary due to factors such as water quality (e.g., alkalinity, hardness), farm cleanliness, and footbath management practices. One of skill in the art will recognize that the amount of concentrate added can vary according to application and need.

As another example, a concentrated antimicrobial composition activated by the activator composition can include: about 35% to about 45% water; about 45% to about 55% sulfuric acid; and about 10% to about 20% ammonium sulfate (e.g., Tasker Saniphresh™, Step 1 footbath). In a preferred embodiment, the antimicrobial composition includes 52.2% by weight of 93% sulfuric acid; 36.2% by weight water; and 11.6% by weight ammonium sulfate. As a further example, a concentrated antimicrobial composition activated by the activator composition can include: about 50% to about 70% water; about 15% to about 25% sulfuric acid; about 5% to about 15% copper sulfate; about 1% to about 5% zinc sulfate; about 0.1% to about 1% sodium chloride; about 0.1% to about 1% potassium chloride; and about 0.1 to about 1% ammonium sulfate (e.g., Tasker SaniPhresh™, Step 2 footbath). In various embodiments, the Tasker SaniPhresh™ Step One and Two solutions, or similar solutions, can be used in conjunction. In some embodiments, a concentrated antimicrobial composition contains about 25-50% water, about 45-65% sulfuric acid, and about 5-25% ammonium sulfate (e.g., Tasker Unifresh® Xtra). In various embodiments, the Tasker SaniPhresh™ Step One and Two solutions, or similar solutions, can be used in conjunction with Tasker Unifresh® Xtra and FootBAth Concentrate, or similar solutions.

The antimicrobial composition can be a blended composition of ingredients described herein, thus forming a homogenous mixture of the solids. The antimicrobial composition can be packaged in dry blended form (e.g., packaged in about 10 lb-about 50 lb bags). The dry blended form can be directly applied to the foot of an animal as a powder or dust. The dry blended form can be mixed with water to form a concentrated antimicrobial composition. The dry blended form can be mixed with water to form a dilute use antimicrobial composition.

Kit

In various embodiments, the present invention can also involve kits for use in the treatment of an infectious disease of a foot of an animal. Such kits can include the compositions of the present invention and, in certain embodiments, instructions for administration and/or usage. When supplied as a kit, the different components of the composition can be packaged in separate containers and admixed immediately before or during use. Such packaging of the components separately can, if desired, be presented in a pack or dispenser device which may contain one or more unit dosage forms containing the composition. Such packaging of the components separately can also, in certain instances, permit long-term storage without losing activity of the components. In various embodiments, the different components can be packaged in one composition for administration together.

In certain embodiments, kits can be supplied with instructional materials. Instructions may be printed on paper or other substrate, and/or may be supplied as an electronic-readable medium, such as a floppy disc, mini-CD-ROM, CD-ROM, DVD-ROM, Zip disc, videotape, audio tape, and the like. Detailed instructions may not be physically associated with the kit; instead, a user may be directed to an Internet web site specified by the manufacturer or distributor of the kit.

Method of Treatment

Another aspect of the invention is directed towards treatment of a foot of an animal by administration of one or more of the compositions described herein. Various embodiments are directed towards treatment of an infectious disease of a foot of an animal by administration of one or more of the compositions described herein. Infectious diseases of the foot of an animal that can be treated with compositions and methods described herein include, but are not limited to, hairy heel warts, foot rot, and/or scald. Infectious diseases of the foot of an animal that can be treated with compositions and methods described herein include, but are not limited to conditions caused by Fusobacterium necrophorum, Bacteroides melaminogenicus, and/or Diechelobacter nodosus. As used herein, treatment is generally understood to encompass both prophylactic treatment as well as treatment of an existing or diagnosed condition. For example, an animal in need of treatment can be at risk, or determined to be at risk, for an infection of the foot. As another example, an animal in need of treatment can have, or be diagnosed as having, an infection of the foot. Diagnosis and risk assessment for animal foot diseases discussed herein is within the skill of the art.

In some embodiments, the method for treating an infectious disease of a foot of an animal comprises contacting a foot of an animal in need thereof with an antimicrobial composition described herein (e.g., an antimicrobial composition comprising water, sulfuric acid, ammonium sulfate or sodium sulfate, and optionally an antimicrobial metal). In some embodiments, the method further comprises mixing a concentrated antimicrobial composition and water to form an antimicrobial composition for contacting a foot of an animal. In various configurations, a concentrated antimicrobial composition, copper sulfate, and water are mixed to form an antimicrobial composition for contacting a foot of an animal.

An antimicrobial composition can be administered in the same or different form as a single dose or in multiple applications. In some embodiments, a first antimicrobial composition and a second antimicrobial composition are applied serially to the foot of an animal. In some embodiments, a first concentrated antimicrobial composition is diluted with water to form a first antimicrobial composition, and a second concentrated antimicrobial composition is diluted with water to form a second antimicrobial composition. The first and the second antimicrobial composition can be applied serially or jointly to a foot of an animal at predetermined intervals, as described further herein.

Subject

In various embodiments, the animal to be treated is an ungulate. An ungulate is understood to include an animal having hooves, or feet resembling hooves, or feet that are hoof-like. An ungulate is also understood to include an animal of, or belonging to, the former order Ungulata, now divided into the orders Perissodactyla and Artiodactyla and composed of hoofed mammals such as, but not limited to, a horse, a cow, a goat, a sheep, a pig, deer, an elephant, an elk, a bison, a moose, a gazelle, and an antelope. Preferably, the subject of various treatments described herein is a cow, a sheep, a horse, or a goat. More preferably, the ungulate to be treated with an antimicrobial composition of the present invention is a cow. Infectious diseases in the hooves of cows are most prevalent in dairy cow herds but are also problematic in beef cattle. The treatment compositions and methodologies described herein can be directed to dairy and/or beef cattle. In some embodiments, the ungulate to be treated with an antimicrobial composition of the present invention is a goat. Research has shown that some goats (e.g., Boer goats) are more prone to foot rot than others. In some embodiments, the ungulate to be treated with an antimicrobial composition of the present invention is a horse. In some embodiments, the ungulate to be treated with an antimicrobial composition of the present invention is a sheep. Research has also shown that some breeds of sheep (e.g., Merino sheep) are more susceptible to foot rot and foot scald than other breeds.

Need

The compositions and methods described herein, used alone or in combination with other known treatment compositions and modalities, can be directed to the treatment of an infectious disease of the foot of an animal. Such infectious diseases of the foot of an animal include, but are not limited to, hairy heel warts, foot rot, and/or scald. Foot rot includes, but is not limited to, stable foot rot.

Generally, an animal is in need of treatment when an animal is diagnosed with, or at risk for, an infectious disease of the foot. As an example, an animal can be in need of treatment with the compositions described herein when the animal is diagnosed with hairy heel warts, foot rot, and/or scald. As another example, an animal can be in need of treatment with the compositions described herein when the animal is determined at risk for hairy heel warts, foot rot, and/or scald. Diagnosis of infectious diseases of the foot in animals is within the skill of the art. The determination of risk for an infectious disease of the foot can be according to environmental conditions, susceptibility of certain types of animals, or other factors known to one of skill in the art.

Application

An animal foot can be contacted with an antimicrobial composition described herein by any method or apparatus suitable for applying the antimicrobial agent. For example, an antimicrobial composition can be administered by a method such as a footbath, powder, spray, gel, foam, or a combination thereof. Various embodiments provides for components of the footbath, spray, gel or foam form of the antimicrobial composition to be administered sequentially. In other embodiments of the present invention, a footbath, spray, gel or foam form of an antimicrobial composition can be administered sequentially with an antimicrobial composition in the same or another form such as a footbath, powder, spray, gel, or foam.

Various embodiments provide for the administration of an antimicrobial composition in any combination of a regimen in the control of an infectious disease of a foot of an animal. For example, for treatment and/or control of a disease of the hoof of an animal, an antimicrobial composition can be administered as a footbath, administered as a powder, administered as a spray or a gel, or a combination thereof in various orders of administration. Duration of treatment can be according to a predetermined duration, which can involve multiple applications, or continued over a period of time until determined that treatment is no longer needed. An antimicrobial composition can be administered as a single dose or in multiple applications. Different formulations of the antimicrobial composition can be administered serially during the same treatment session or in different treatment sessions.

An antimicrobial composition can be administered in the same or different form as a single dose or in multiple applications. For example, a footbath may be administered weekly (or several times during the week) as a regimen or may be administered for different times as a regimen.

In various embodiments, an antimicrobial composition is contacted with an animal's feet for a certain minimal contact time so as to provide for an antimicrobial effect. The contact time can vary with concentration of the use composition, method of applying the use composition, temperature of the use composition, the use composition itself (e.g., whether used alone or in conjunction with additional antimicrobial footbath products), and other similar factors that will be apparent to one of skill in the art. Preferably the exposure time is a time that allows at least partial absorption of the antimicrobial solution into/onto the foot/hoof.

An antimicrobial composition described herein can be administered as a vented densified fluid composition, a spray of the agent, by immersion in the agent as in a footbath, by foam or gel treating with the agent, or the like, or any combination thereof. Contact with a spray, a foam, a gel, or by immersion in a liquid or immersion in a powder or other form of the antimicrobial compositions can be accomplished by a variety of methods known to those of skill in the art.

Preferably, an activated antimicrobial composition is administered as a footbath. An animal's foot can be exposed to a footbath containing an antimicrobial composition described herein by a variety of means known to the art, which include, for example, a trough. Another option application via footbath is the use of absorptive pads saturated with the composition to be used for treatment. The pad can be placed in a high traffic area that an animal must pass through, such as a gate, lane, or water trough. When an animal stands to drink at the water, feet can be treated.

In various embodiments, an antimicrobial composition described herein can be administered as a spray. An antimicrobial composition can be applied using fixed or articulating nozzles, at higher pressures, varying or steady flow rates, various temperatures, and/or with or without agitation or brushes. Spraying can be accomplished by an apparatus such as a spray cabinet with stationary or moving spray nozzles. The nozzles can create a mist, vapor, or spray that contacts an animal's feet. The spray can be set up as a walk-through pen or in a holding pen.

Application of a material by spray can be accomplished, for example, using a manual spray wand application, an automatic spray of the animals moving through a gate or room or gateway, or the like. Multiple spray heads to ensure complete contact or other spray means may be used. One preferred automatic spray application involves the use of a spray booth. The spray booth substantially confines the sprayed composition to within the parameter of the booth. The spray booth can include steam jets that can be used to apply an antimicrobial compositions of the invention. The spray pattern can be virtually any useful spray pattern.

In various embodiments, an antimicrobial composition described herein can be administered as a foam. The foam can be prepared, for example, by mixing foaming surfactants with the antimicrobial composition. The foaming surfactants can be nonionic, anionic, or cationic in nature. Examples of useful surfactant types include, but are not limited to the following: alcohol ethoxylates, alcohol ethoxylate carboxylate, amine oxides, alkyl sulfates, alkyl ether sulfate, sulfonates, quaternary ammonium compounds, alkyl sarcosines, betaines, and alkyl amides. The foaming surfactant is typically mixed at time of use with the antimicrobial composition but can be prepared in advance of the time of use. At time of use, compressed air can be injected into the mixture, and the foam can be applied to a foot of an animal. Thickeners (e.g., xanthan gum, polymeric thickeners, cellulose thickeners, propylene glycol, glycerin, or the like) can be further combined to produce a foam which may remain in contact with the infected area of the foot for a longer period of time than a formulation without the thickener. In some embodiments, a foam antimicrobial composition contains about 5 wt % to about 20 wt % of thickeners. As an example, a foam antimicrobial composition can contain about 5 wt % to about 10 wt % propylene glycol and about 5 wt % to about 10 wt % glycerin.

In various embodiments, an antimicrobial composition described herein can be administered as a gel. The animal foot can be treated with a thickened or gelled version of an antimicrobial composition. In the thickened or gelled state, the antimicrobial composition can remain in contact with the animal's foot for longer periods of time, thus increasing the antimicrobial efficacy. The thickened or gelled solution can also better adhere to vertical surfaces and crevices in the animal's foot. The composition can be thickened or gelled using thickening agents including, but not limited to, xanthan gum, polymeric thickeners, cellulose thickeners, propylene glycol, glycerin, or the like. Rod micelle forming systems such as amine oxides and anionic counter ions could also be used. The thickeners or gel forming agents can be used, for example, in the concentrated product or by mixing with the antimicrobial composition at time of use. Exemplary use levels of thickeners or gel agents can range from about 100 ppm to about 10 wt-%.

Immersing an animal's foot in a liquid or powder disinfectant, of the present invention, can be accomplished by any of a variety of methods known to those of skill in the art. For example, troughs can be used to immerse the feet. A disinfectant composition of the present invention, contained in the trough, can be agitated so as to increase the application and/or absorption of the solution into or onto the feet. Agitation can be obtained by conventional methods, including ultrasonics, aeration by bubbling air through the solution, by mechanical methods, such as strainers, paddles, brushes, pump driven liquid jets, or by combinations of these methods. The disinfection agent can be heated to increase the efficacy of the solution in killing microorganisms. After the foot has been immersed for a time sufficient for the desired effect, the foot can be removed from the bath and the antimicrobial composition can be rinsed, drained, blotted, or evaporated from the foot. Treatments with any of the compositions of the present invention can be applied one time or repeatedly within a short period of time (minutes or hours), or the treatments can be repeated as needed over a longer period of time (days or months).

Contact Time

The antimicrobial compositions described herein can be contacted with an animal foot/hoof in an amount effective to result in a reduction significantly greater than is achieved by washing with water, or at least a 50% reduction, preferably at least a 90% reduction, more preferably at least a 99% reduction, in the resident microbial preparation.

The present methods usually require a certain minimal contact time of the composition with the foot/hoof of an animal for occurrence of significant disinfection effect. The contact time can vary with concentration of the use composition, method of applying the use composition, temperature of the use composition, amount of soil and/or contamination on the hoof and/or in the footbath, number of microorganisms present on the hoof, type and formulation of the disinfection composition, or the like. The minimum exposure time is, for example, at least about 2 to about 5 seconds. The exposure time can be, for example, at least about 5 seconds, at least about 10 seconds, at least about 15 seconds, at least about 30 seconds, at least about 45 seconds, at least about one minute, at least about two minutes, at least about three minutes, at least about four minutes, at least about five minutes, at least about six minutes, at least about seven minutes, at least about eight minutes, at least about nine minutes, at least about ten minutes, at least about 15 minutes, at least about 20 minutes, at least about 25 minutes, at least about 30 minutes, at least about 35 minutes, at least about 40 minutes, at least about 45 minutes, at least about 50 minutes, at least about 55 minutes, or at least about 60 minutes. Even longer exposure times are contemplated, for example, several hours or even days. Generally, longer exposure times to the disinfection composition will be preferred as the pH increases and/or the antimicrobial metal (e.g., copper) content decreases. After direct exposure during administration, it is contemplated that the disinfection composition can remain in contact with a hoof for extended periods of time.

Application of an antimicrobial composition (and/or a series of different antimicrobial compositions) to an animal's foot can occur in periodic sequential applications. An effective amount of antimicrobial composition can be applied to an animal's foot several times per day and/or several times over a period of several days. For example, the disinfection composition can be applied to part or all of an animal's foot/hoof about every one hour, about every two hours, about every three hours, about every four hours, about every five hours, about every six hours, about every seven hours, about every eight hours, about every nine hours, about every ten hours, about every eleven hours, or about every twelve hours. Longer periods of time between applications are contemplated. For example, the antimicrobial composition can be applied every day or every several days.

Having described the invention in detail, it will be apparent that modifications, variations, and equivalent embodiments are possible without departing the scope of the invention defined in the appended claims. Furthermore, it should be appreciated that all examples in the present disclosure are provided as non-limiting examples.

### EXAMPLES

The following non-limiting examples are provided to further illustrate the present invention. It should be appreciated by those of skill in the art that the techniques disclosed in the examples that follow represent approaches the inventors have found function well in the practice of the invention, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the invention.

### Example 1

### Footbath Studies

The following example compares compositions of the present invention to conventional describes use of rotational footbath programs with Tasker SaniPhresh™ Step 1 and Tasker SaniPhresh™ Step 2 in combination with reduced amounts of copper sulfate as a replacement for rotational footbath programs using copper sulfate and formaldehyde at controlling hoof lesions on large dairy herds. The objective of the trials was to compare the effectiveness of the Tasker SaniPhresh™ Step 1 and Tasker SaniPhresh™ Step 2 Rotational Footbath Program with a copper sulfate and formaldehyde rotational program at controlling hoof lesions (Hairy Heel Warts and Foot Rot) on two large commercial dairy herds.

A footbath program using footbath solutions made up with copper sulfate crystals in rotation with milking shifts using footbath solutions made up with formaldehyde is historically considered an effective hoof care program. In this example, a Tasker SaniPhresh™ Step 1 and Step 2 Rotational Program is compared to a conventional copper sulfate/formaldehyde program to assess hoof lesion protecting properties.

Two Michigan dairy farms were selected for the trial. The farms had well managed footbath programs and the herds were not experiencing a hoof condition outbreak. Both farms used rotational footbath solutions using copper sulfate and formaldehyde. The farm trials were for an eight-week period. A Tasker SaniPhresh™ Step 1 and Step 2 Rotational Program replaced the previous copper sulfate/formaldehyde footbath programs. All hoofs in the herds were evaluated and scored at the beginning and at the end of the trial. Cows with one or more hoof lesions (Hairy Heel Warts or Foot Rot) were documented.

For farm A, the herd size was 2,400 cows. The dairy farm A uses two 40-gallon footbaths. The conventional footbath program of farm A consisted of using: 25 pounds of copper sulfate per bath, 4 days per week; 2 gallons formaldehyde per bath, 1 day per week; where the footbath was changed at 400 cows and footbaths were not used 2 days per week. The trial footbath program at farm A consisted of using: 32 oz. Tasker SaniPhresh™ Step 1 plus 10 pounds copper sulfate per bath, 2 days per week; 32 oz. Tasker SaniPhresh™ Step 2 plus 10 pounds copper sulfate per bath, 3 days per week; where the footbath was changed at 400 cows and footbaths were not used 2 days per week.

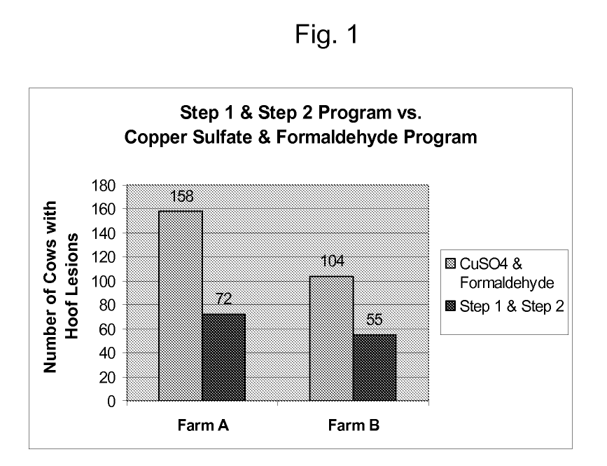

Results from farm A showed that, at the start of the trial (i.e., under copper sulfate and formaldehyde program), 158 cows had hoof lesions, while at the end of the trial (i.e., after the Tasker SaniPhresh™ program), 72 cows had hoof lesions, for a 54% reduction in cows with hoof lesions (see e.g., FIG. 1).

For farm B, the herd size was 3,000 cows. The dairy farm B uses two 125-gallon footbaths. The conventional footbath program of farm B consisted of using: 25 pounds of copper sulfate per bath, 3 days per week; 5 gallons formaldehyde per bath, 3 days per week; where the footbath was changed at 350 cows and footbaths were not used 1 day per week. The trial footbath program at farm B consisted of using: 80 oz. Tasker SaniPhresh™ Step 1 plus 25 pounds copper sulfate per bath, 3 days per week; 96 oz. Tasker SaniPhresh™ Step 2 plus 25 pounds copper sulfate, 3 days per week; where the footbath was changed at 500 cows and footbaths were not used 1 day per week.

Results from farm B showed that, at the start of the trial (i.e., under copper sulfate and formaldehyde program), 104 cows had hoof lesions, while at the end of the trial (i.e., after the Tasker SaniPhresh™ program), 55 cows had hoof lesions, for a 47% reduction in cows with hoof lesions.

In both studies, the SaniPhresh™ Step 1 and Step 2 Rotational Footbath Program: eliminated the use of formaldehyde, which creates serious personnel safety issues, on the farm; reduced the amount of copper sulfate use, which entails manure, land residue, and cost issues; and lowered the number of footbath changes, which saves time, water, chemicals.

In conclusion, the SaniPhresh™ Step 1 and Step 2 Rotational Footbath Program is an effective footbath program at controlling the incidence of hoof lesions in a dairy herd and the program is significantly more effective than conventional footbath programs. Compared to the number of cows having hoof lesions on the conventional copper sulfate/formaldehyde programs, the number of cows with hoof lesions after 8 weeks on the STEP 1 and STEP 2 Program was reduced by approximately 50%.

### Example 2

### Footbath Studies

The following example demonstrates the influence of Tasker SaniPhresh™ Step 1 and 2 Rotational Footbath Program on the incidence of heel warts (Papillomatosis Digital Dermatitis, PDD) in commercial dairy herds from two farms, C and D.

The commercial dairy farm C had 550 cows, which were milked three times daily. Prior to the study, farm C had been unsuccessful in controlling heel warts with prior footbath protocols. Over 20% of the herd from farm C was diagnosed as having heel warts prior to the study. Heel wart-related pain is positively associated with lameness, involuntary culling, reduced food and water intake, reduced milk production, and reduced profits. The study was conducted over 8 weeks, with 5 evaluations conducted at 2 week intervals along with weekly monitoring.

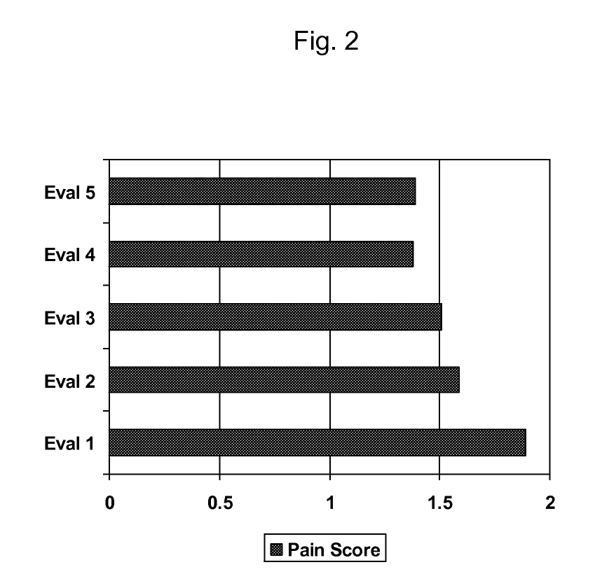

Results from farm C showed that the Tasker SaniPhresh™ Step 1 and Step 2 foot bath program significantly reduced the severity of PDD (heel wart) pain regardless of pen over the 8 weeks of the trial (see e.g., FIG. 2). Hoof trimmer, herd manager and farm manager at farm C reported a reduction in wart severity as measured by hoof trimmer examination and treatment of cows trimmed.

Examination of weekly hoof trimmer records from farm C show an overall reduction in the percentage of cows wrapped and/or trimmed during the seven weeks of the trial as compared to the seven weeks immediately preceding the trial (see e.g., Table 1). An increase in warts seen during initial 2 weeks of trial, which was thought to be due to the dairy's use of soap and salt in footbaths 3-4 weeks prior to trial.

TABLE 1
Pre-trial hoof trimmer records from farm C

| | # cows | | % cows |
|---|---|---|---|
| Week | trimmed | # wraps | Wrapped |
| 1 | 50 | 14 | 28 |
| 2 | 52 | 18 | 35 |
| 3 | 49 | 13 | 26.5 |
| 4 | 49 | 18 | 37 |
| 5 | 48 | 17 | 35 |
| 6 | 57 | 20 | 35 |
| 7 | 49 | 13 | 26.5 |

TABLE 2
Trail hoof trimmer records from farm C (SaniPhresh ™ Step
1 and Step 2)

| | START OF TRIAL | | |
|---|---|---|---|
| 1 | 53 | 26 | 49 |
| 2 | 101 | 44 | 43 |
| 3 | 54 | 8 | 15 |
| 4 | 52 | 11 | 21 |
| 5 | 51 | 7 | 14 |
| 6 | 41 | 11 | 27 |
| 7 | 43 | 10 | 23 |

The commercial dairy farm D had 830 milking cows. Prior to the study, farm D had been unsuccessful in controlling heel warts with prior copper sulfate footbath protocols. Over 30% of the herd from farm D was diagnosed as having heel warts prior to the study, as diagnosed by hoof trimmer assessments and lesions on the heel and interdigital tissue. The study was conducted over 8 weeks.

Results showed that the incidence of heel warts and interdigital dermatitis was reduced from greater than 30% to less than 5% of cows trimmed. After continued use over more than an additional month, less than 1% of cows in the herd were reported to have heel warts.

The commercial dairy farm D had 830 milking cows. Prior to the study, farm D had been unsuccessful in controlling heel warts with prior copper sulfate footbath protocols. Over 30% of the herd from farm D was diagnosed as having heel warts prior to the study, as diagnosed by hoof trimmer assessments and lesions on the heel and interdigital tissue. The study was conducted over 8 weeks.

In conclusion, Tasker Sainifresh Step 1 and Step 2 footbath concentrates were shown to be effective in controlling the incidence of, and pain associated with, heel warts (PDD) when used as part of a well managed footbath program.

### Example 3

### Disinfection Composition Effect on Listeria Monocytogenes

The effect of an acidic buffered copper containing disinfection composition was examined on Listeria monocytogenes. The disinfection agent was commercially available Tasker Pacific Blue (including sulfuric acid, ammonium sulfate, copper sulfate, and water).

Twelve tubes of nutrient broth were inoculated with 10 ul of culture (L. mono., Scott A strain, log phase growth at 10⁸ cfu/ml) to contain 10⁵ cfu/ml Listeria monocytogenes. 1 ml of peptone buffer is added to the control samples (6). 1 ml of Tasker 10× solution (pH 2.8, 3 ppm copper) to treated samples (6). Tubes stored at 40° F. for 4 days and then analyzed for Listeria monocytogenes.

Results showed that controls had 3,000,000 cfu/ml at Day 1 and 21,000,000 cfu/ml at Day 7. Treated groups had 200 cfu/ml at Day 1 and <10 cfu/ml at Day 7 (see e.g., FIG. 15; Table 8).

TABLE 8
Tasker Blue effect on L. Mono content
After 24 hours, plated on Palcam Agar:

| | | | |
|---|---|---|---|
| 40° F. | control | 3,000,000 | 6.477121 |
| | treated | 200 | 2.30103 |
| 45° F. | control | 3,000,000 | 6.477121 |
| | treated | 1,100 | 3.041393 |

After 4 Days:

| 40° F. | control | 21,000,000 | | 7.322219 | |
|---|---|---|---|---|---|
| | treated | 10 | | 1 | 0.7 |
| 45° F. | control | 43,000,000 | cfu/ml | | |
| | treated | <10 | cfu/ml | | |

### Example 4

### Disinfection Composition Effect on E. coli

The inhibitory activity of acidic buffered copper-containing disinfection agents was determined against Escherichia coli ATCC 11229. The disinfection agent was commercially available Tasker Blue (including sulfuric acid, ammonium sulfate, copper sulfate pentahydrate, and water).

Test samples were prepared for testing at pH levels of 2.0, 2.5, 3.0, 3.5, and 4.0 in combination with copper concentrations of 0 ppm, 1 ppm, 2 ppm, and 3 ppm. Tryptic soy Broth was prepared half strength as a standard inoculum of 0.5 McFarland. The test sample was added to a sterile tube, along with the same amount of standardized Escherichia coli ATCC 11229 inoculum. The pH of the sample was recorded and adjusted as indicated on the test sample bottle. Tubes were incubated for 24 hours at 35° C. and the inhibitory concentration was determined as the lowest concentration showing visible inhibition of the growth of the organism. All samples were run in duplicate along with positive and negative growth controls. Final pH of test samples were recorded following completion of 24 hour incubation.

Results showed that complete inhibition of microbial growth was achieved with all solutions except the following solutions, in which microbial growth was detected: pH 4.0 Cu 0 ppm; pH 4.0 Cu 1 ppm; pH 4.0 Cu 2 ppm; pH 4.0 Cu 3 ppm.

### Example 5

### Disinfectant Formulations

The inhibitory activity of acidic buffered disinfection agents on aerobic plate count (APC) was examined. Five formulations were tested.

Mark I: a 24 hour high temperature reaction process at approximately 300-350° F. with a stabilization step after overnight cooling. Composed of reacting 98% sulfuric acid with a 26-28% by weight ammonium sulfate in water solution. The order of addition was ammonium sulfate solution to sulfuric acid. Electrolysis of the reacting solution was applied for 1 hour at the start of the process. The stabilization step was the addition of more ammonium sulfate solution to ensure that the reaction is complete. The Tasker Clear™ product formed was a buffered acid solution of a strong acid (sulfuric acid) and a salt (ammonium sulfate) of a strong acid and strong base.

Mark II: a 2 hour low temperature reaction process at approximately 200-210° F. with a stabilization step immediately after the 1 hour electrolysis period. This was the same process as in the Mark I product above except that it was performed at a lower temperature and a shorter period of time. The ingredient amounts were adjusted to account for no lost of water as was seen in the Mark I process. The Tasker Clear™ product formed was a buffered acid solution of a strong acid (sulfuric acid) and a salt (ammonium sulfate) of a strong acid and strong base.

Mark III: a low temperature reaction process in which the 98% sulfuric acid was added slowly to a 30% by weight ammonium sulfate solution. The addition was done continuously until all the ammonium sulfate solution was added. There was no stabilization step. The addition order was the reverse of the Mark I, II, IV, and V processes. The temperature was maintained in the 150-200° F. range during the addition process. No electrolysis was performed during this process and hence the name ‘cold process’ was given to it. The Tasker Clear™ product formed was a buffered acid solution of a strong acid (sulfuric acid) and a salt (ammonium sulfate) of a strong acid and strong base.

Mark IV: a 4 hour high temperature reaction process at approximately 300-350° F. with a stabilization step after cooling. Composed of reacting 98% sulfuric acid with a 26-28% by weight sodium sulfate in water solution. The order of addition was sodium sulfate solution to sulfuric acid. Electrolysis of the reacting solution was applied for 1 hour at the start of the process. The stabilization step was the addition of more sodium sulfate solution to ensure that the reaction is complete. The Tasker Clear™ product formed was a buffered acid solution of a strong acid (sulfuric acid) and a salt (sodium sulfate) of a strong acid and strong base. (Note: In this process sodium sulfate was substituted for ammonium sulfate.)

Mark V: a 4 hour high temperature reaction process at approximately 300-350° F. with a stabilization step after cooling. Composed of reacting 98% sulfuric acid with a 26-28% by weight sodium sulfate in water solution. The order of addition was sodium sulfate solution to sulfuric acid. There was no electrolysis during this process (cold process). The stabilization step was the addition of more sodium sulfate solution to ensure that the reaction was complete. The Tasker Clear™ product formed was a buffered acid solution of a strong acid (sulfuric acid) and a salt (sodium sulfate) of a strong acid and strong base. (Note: In this process sodium sulfate was substituted for ammonium sulfate, and no electrolysis was performed.)

Results showed that all formulations exponentially reduced the aerobic plate count (see e.g., Table 3).

TABLE 3

| | | |
|---|---|---|
| | Counts | Log₁₀ |
| Time | cfu/ml | cfu/ml |

Butterfield Buffer Control

| | | |
|---|---|---|
| 0 | 845 | 2.93 |
| 5 | 780 | 2.89 |
| 15 | 785 | 2.89 |
| | | Ave = 2.90 |

DI Water Control

| | | |
|---|---|---|
| 0 | 1015 | 3.01 |
| 5 | 1075 | 3.03 |
| 15 | 940 | 2.97 |
| | | Ave = 3.00 |

| | | | |
|---|---|---|---|
| | Counts | Log₁₀ | Log |
| Time | cfu/ml | cfu/ml | Reduction |

Mark I Solution

| | | | |
|---|---|---|---|
| 0 | 140 | 2.15 | 0.85 |
| 5 | 25 | 1.40 | 1.60 |
| 15 | 5 | 0.70 | 2.30 |

Mark II Solution

| | | | |
|---|---|---|---|
| 0 | 100 | 2.00 | 1.00 |
| 5 | 30 | 1.48 | 1.52 |
| 15 | 0 | 0.00 | 3.00 |

Mark III Solution

| | | | |
|---|---|---|---|
| 0 | 65 | 1.81 | 1.19 |
| 5 | 0 | 0.00 | 3.00 |
| 15 | 0 | 0.00 | 3.00 |

Mark IV Solution

| | | | |
|---|---|---|---|
| 0 | 110 | 2.04 | 0.96 |
| 5 | 40 | 1.60 | 1.40 |
| 15 | 0 | 0.00 | 3.00 |

Mark V Solution

| | | | |
|---|---|---|---|
| 0 | 125 | 2.10 | 0.90 |
| 5 | 20 | 1.30 | 1.70 |
| 15 | 5 | 0.70 | 2.30 |

NOTES:
* Log Reduction based on DI Water average log₁₀ = 3.00
** Counts are the average of duplicate APC plates

What is claimed is:

1. A method for treating a foot of an animal, the method comprising contacting a foot of an animal with an antimicrobial composition comprising (a) water, (b) sulfuric acid, and (c) ammonium sulfate or sodium sulfate.

2. The method of claim 1, wherein the antimicrobial composition comprises water, sulfuric acid, and ammonium sulfate.

3. The method of claim 1, wherein the antimicrobial composition comprises water, sulfuric acid, and sodium sulfate.

4. The method of claim 1, wherein the antimicrobial composition further comprises an effective amount of at least one antimicrobial metal.

5. The method of claim 4, wherein the antimicrobial metal is copper, zinc, magnesium, silver, or iron.

6. The method of claim 5, wherein the antimicrobial metal is copper.

7. The method of claim 5, wherein the antimicrobial metal is zinc.

8. The method of claim 4, wherein the antimicrobial composition further comprises an effective amount of at least two antimicrobial metals.

9. The method of claim 8 wherein the antimicrobial composition comprises copper and zinc.

10. The method of claim 1, wherein the antimicrobial composition comprises water, sulfuric acid, ammonium sulfate, and copper sulfate.

11. The method of claim 1, wherein the antimicrobial composition comprises water, sulfuric acid, sodium sulfate, and copper sulfate.

12. The method of claim 4, wherein the antimicrobial metal concentration is greater than about 1 ppm.

13. The method of claim 12, wherein the antimicrobial metal concentration is about 1 ppm to about 4,000 ppm.

14. The method of claim 13, wherein the antimicrobial metal concentration is about 10 ppm to about 300 ppm.

15. The method of claim 14, wherein the antimicrobial metal concentration is about 15 ppm to about 150 ppm.

16. The method of claim 15, wherein the antimicrobial composition is a foam and the antimicrobial metal concentration is about 500 ppm to about 2,000 ppm.

17. The method of claim 16, wherein the antimicrobial composition is a foam and the antimicrobial metal concentration is about 1,000 ppm.

18. The method of claim 1, wherein the pH of the antimicrobial composition is about 0.5 to about 5.0.

19. The method of claim 18, wherein the pH of the antimicrobial composition is about 1.0 to about 4.5.

20. The method of claim 19, wherein the pH of the antimicrobial composition is about 1.5 to about 2.0 at the beginning of the treatment, the feet of at least about 300 animals are contacted with the antimicrobial composition, and the pH of the antimicrobial composition after contact with the feet of the at least about 300 animals is not more than about 4.5.

21. The method of claim 20, wherein the pH of the antimicrobial composition is about 1.5 to about 2.0 at the beginning of the treatment, the feet of at least about 400 animals are contacted with the antimicrobial composition, and the pH of the antimicrobial composition after contact with the feet of the at least about 400 animals is not more than about 4.5.

22. The method of claim 21, wherein the pH of the antimicrobial composition is about 1.5 to about 2.0 at the beginning of the treatment, the feet of at least about 500 animals are contacted with the antimicrobial composition, and the pH of the antimicrobial composition after contact with the feet of the at least about 500 animals is not more than about 4.5.

23. The method of claim 1, wherein the antimicrobial composition comprises water, sulfuric acid, ammonium sulfate, and copper sulfate, and the antimicrobial composition has a pH of about 1.5 to about 4.0.

24. The method of claim 1, wherein the antimicrobial composition comprises water, sulfuric acid, sodium sulfate, and copper sulfate, and the antimicrobial composition has a pH of about 1.5 to about 4.0.

25. The method of claim 1, further comprising the step of mixing a concentrated antimicrobial composition and water to form the antimicrobial composition.

26. The method of claim 25 wherein the mixed antimicrobial composition comprises at an effective amount of at least one antimicrobial metal selected from the group consisting of copper, zinc, magnesium, silver, or iron at a concentration of about 10 ppm to about 300 ppm.

27. The method of claim 6, further comprising the step of mixing a concentrated antimicrobial composition, copper sulfate, and water to form the antimicrobial composition.

28. The method of claim 27 wherein the mixed antimicrobial composition has a copper concentration of about 1,000 ppm to about 4,000 ppm.

29. The method of claim 1, wherein the animal is an ungulate.

30. The method of claim 29, wherein the ungulate is a cow.

31. The method of claim 30, wherein the cow is a dairy cow.

32. The method of claim 1, wherein contacting comprises bathing, spraying, or dipping.

33. The method of claim 1, wherein in the antimicrobial composition is a liquid, gel, or foam.

34. The method of claim 1, wherein the animal is diagnosed with, or at risk for, an infectious disease of the foot.

35. The method of claim 34, wherein the infectious disease of the foot is selected from the group consisting of hairy heel warts, foot rot, and foot scald.

36. A method for treating a foot of an animal, the method comprising:
mixing a first concentrated antimicrobial composition and water to form a first antimicrobial composition, wherein the first concentrated antimicrobial composition comprises water, sulfuric acid, and ammonium sulfate or sodium sulfate;
contacting a foot of an animal with the first antimicrobial composition;
mixing a concentrated antimicrobial composition, copper sulfate, and water to form a second antimicrobial composition, wherein the second concentrated antimicrobial composition comprises water, sulfuric acid, and ammonium sulfate or sodium sulfate; and
contacting the foot of the animal with the second antimicrobial composition.
